(19) United States
(12) Patent Application Publication
(10) Pub. No.: US 2025/0255632 A1
(43) Pub. Date: Aug. 14, 2025

(54) CATHETER ASSEMBLIES HAVING A DILATABLE RETRIEVAL BASKET AND METHODS OF USE

(71) Applicant: Bard Peripheral Vascular, Inc., Franklin Lakes, NJ (US)

(72) Inventors: Kyle Hull, Phoenix, AZ (US); Kristen Hunn, Gilbert, AZ (US); Tyler Lent, Mesa, AZ (US); Danielle Worger, Mesa, AZ (US)

(73) Assignee: Bard Peripheral Vascular, Inc., Franklin Lakes, NJ (US)

(21) Appl. No.: 18/857,008

(22) PCT Filed: Jun. 8, 2022
(86) PCT No.: PCT/US2022/032660

§ 371 Date: Oct. 15, 2024

(51) Int. Cl. A61B 17/221; A61B 17/00; A61B 17/22; A61M 25/00; A61M 25/10

(52) U.S. Cl. CPC A61B 17/221; A61B 17/00234; A61M 25/0043; A61M 25/10; A61B 2017/00238; A61B 2017/00305; A61B 2017/00367; A61B 2017/00477; A61B 2017/00836; A61B 2017/00862; A61B 2017/00867; A61B 2017/22065; A61B 2017/2212; A61M 2025/0004; A61M 2210/12

(57) ABSTRACT

A catheter assembly for performing a thrombectomy and methods of use, the catheter assembly including a catheter sheath and a dilation and capture assembly slidably positioned within the catheter sheath. The dilation and capture assembly includes a catheter having a balloon portion at a distal end of the catheter, and a retrieval basket. The balloon portion is selectively inflatable and deflatable between respectively a maceration orientation and a delivery orientation. The retrieval basket is deployable from a delivery configuration mounted to the balloon portion and a retrieving configuration axially displaced from the distal end of the catheter.

### TECHNICAL FIELD

The present specification generally relates to catheter assemblies for performing a thrombectomy and an angioplasty and, more specifically, catheter assemblies having a dilatable retrieval basket and methods of use.

### BACKGROUND

A thrombectomy is performed to reduce the size of or completely dislodge a thrombus in a body vessel of a patient, while an angioplasty is performed to dilate a restricted vessel. The dislodged thrombus may be carried by blood flow in the body vessel to another location of a body of the patient, where the thrombus may cause further health complications. For example, the thrombus may become lodged in the body vessel, thereby reducing or completely restricting blood flow along the body vessel. This may result in health complications such as a stroke. A need for improved catheter assemblies for performing a thrombectomy and an angioplasty in an efficient and effective manner exists.

### SUMMARY

In one embodiment, a catheter assembly for performing an angioplasty, the catheter assembly includes a catheter sheath and a dilation and capture assembly slidably positioned within the catheter sheath. The dilation and capture assembly includes a catheter having a balloon portion at a distal end of the catheter, and a retrieval basket. The balloon portion is selectively inflatable and deflatable between respectively a maceration orientation and a delivery orientation. The retrieval basket is deployable from a delivery configuration mounted to the balloon portion and a retrieving configuration axially displaced from the distal end of the catheter.

In another embodiment, a catheter assembly for performing an angioplasty, the catheter assembly includes a catheter sheath and a dilation and capture assembly slidably positioned within the catheter sheath. The dilation and capture assembly includes a catheter including a proximal portion and a balloon portion at a distal end of the proximal portion, a retrieval basket, and one or more attachment wires. The balloon portion is selectively inflatable and deflatable between respectively a maceration orientation and a delivery orientation. The retrieval basket is deployable from a delivery configuration mounted to the balloon portion and a retrieving configuration axially displaced from the balloon portion. The one or more attachment wires are configured to position the retrieval basket relative to the catheter sheath.

In yet another embodiment, a method of performing an angioplasty, the method includes inserting a catheter assembly into a body vessel, translating a balloon portion and a retrieval basket relative to a catheter sheath past a treatment site; and inflating the balloon portion, thereby dilating the retrieval basket. The catheter assembly includes a catheter sheath and a dilation and capture assembly slidably positioned within the catheter sheath. The dilation and capture assembly includes a catheter including a balloon portion at a distal end of the catheter, and a retrieval basket. The balloon portion is selectively inflatable and deflatable between respectively a maceration orientation and a delivery orientation. The retrieval basket is deployable from a delivery configuration mounted to the balloon portion and a retrieving configuration axially displaced from the distal end of the catheter.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a side view of a catheter assembly for performing an angioplasty disposed in a body vessel and advanced to a thrombus site in a thrombus site position, according to one or more embodiments shown and described herein;

FIG. 2 schematically depicts a side view of a catheter of the catheter assembly of FIG. 1, a balloon portion of the catheter shown in an inflated position, according to one or more embodiments shown and described herein;

FIG. 3 schematically depicts a side view of a retrieval basket of the catheter assembly of FIG. 1 shown in a deployed position, according to one or more embodiments shown and described herein;

FIG. 4 schematically depicts a cross sectional front view of the catheter assembly of FIG. 1 taken along line 4-4, according to one or more embodiments shown and described herein;

FIG. 5 schematically depicts another cross sectional front view of the catheter assembly of FIG. 1 taken along line 5-5, according to one or more embodiments shown and described herein;

FIG. 6 schematically depicts a side view of the catheter assembly of FIG. 1 in a delivery configuration past the thrombus site in a past thrombus position, with the balloon portion deflated and the retrieval basket not deployed, according to one or more embodiments shown and described herein;

FIG. 7 schematically depicts a side view of the catheter assembly of FIG. 6 in the delivery configuration in the past thrombus position, with the balloon portion inflated and the retrieval basket deployed, according to one or more embodiments shown and described herein;

FIG. 8 schematically depicts a side view of the catheter assembly of FIG. 7 in a separated position in the past thrombus position, with the balloon portion deflated and the retrieval basket deployed, according to one or more embodiments shown and described herein;

FIG. 9 schematically depicts a side view of the retrieval basket of FIG. 8 in a retrieving configuration in the past thrombus position with the balloon portion proximally displaced to be at the thrombus site position, according to one or more embodiments shown and described herein;

FIG. 10 schematically depicts a side view of the catheter of FIG. 9 in a deflated delivery orientation after being in an inflated maceration orientation to break thrombus, and the retrieval basket of FIG. 9 in the retrieving configuration, according to one or more embodiments shown and described herein;

FIG. 11 schematically depicts the catheter of FIG. 10 in a balloon removal position in which the balloon portion is proximally advanced from the thrombus site position, according to one or more embodiments shown and described herein;

FIG. 12 schematically depicts the retrieval basket of FIG. 10 in a retrieval basket removal position and retrieving configuration to retrieve thrombus when proximally being advanced back past the thrombus site including broken thrombus, according to one or more embodiments shown and described herein; and

FIG. 13 depicts the catheter assembly of FIG. 12 in an assembly extraction position, according to one or more embodiments shown and described herein.

### DETAILED DESCRIPTION

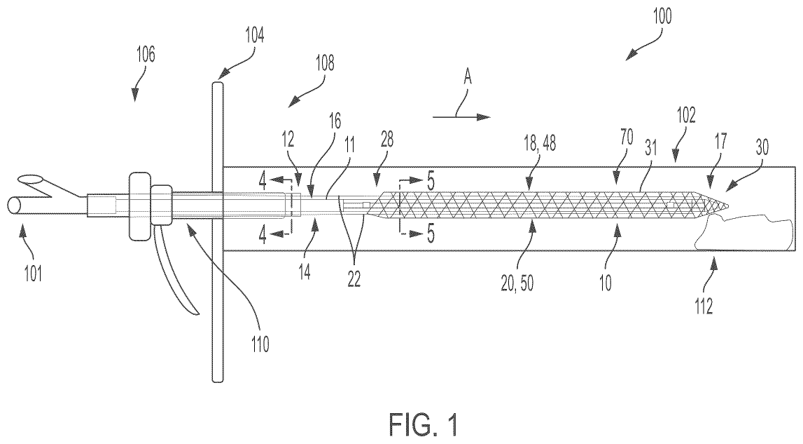

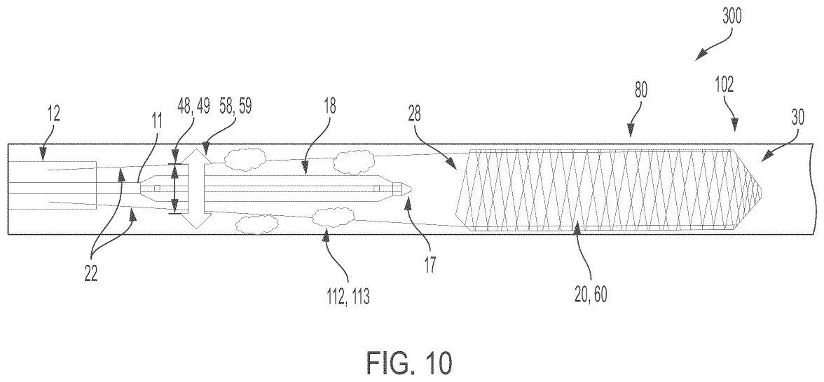

FIG. 1 generally depicts an embodiment of a catheter assembly 10 for performing a thrombectomy disposed in a body vessel 102 and advanced to a thrombus site position 100 at a thrombus 112 as a treatment site of a patient. The catheter assembly 10 may simultaneously perform an angioplasty by using a balloon catheter for both the thrombectomy and the angioplasty, as will be described in further detail herein. An angioplasty, as used herein, is a procedure for dilating a restricted blood vessel. It is contemplated that the “patient” described herein may be a human or an animal. The catheter assembly 10 of FIG. 1 includes a catheter sheath 12 and a dilation and capture assembly 14 slidably positioned within the catheter sheath 12 to allow the dilation and capture assembly 14 to move relative to the catheter sheath 12. The dilation and capture assembly 14 includes a catheter 16 including a balloon portion 18 configured to be inflatable and deflatable between respectively a maceration orientation 59 (FIG. 10) and a delivery orientation 49 (FIG. 10) to macerate the thrombus 112 at a treatment site. The dilation and capture assembly 14 further includes a retrieval basket 20 that is movable relative to the balloon portion 18 so that the retrieval basket 20 is positionable downstream of the balloon portion 18 to be distally and axially displaced from the balloon portion 18 in a retrieving configuration 80 (FIG. 10), as described in greater detail further below. The retrieval basket 20 is configured to collect particulate removed from the thrombus 112 as broken thrombus 113 during a maceration operation of the balloon portion 18 as described herein. Various embodiments of the catheter assembly and the operation of the catheter assembly will be described in more detail herein.

The catheter assembly 10 of FIG. 1 may be distally moved in the direction of arrow A to perforate a body 104 of the patient and be inserted and move distally into the body vessel 102. The catheter assembly 10 may include an introducer sheath 110, the catheter sheath 12, and the dilation and capture assembly 14 slidably positioned within the catheter sheath 12 so that the dilation and capture assembly 14 may move relative to the catheter sheath 12. The introducer sheath 110 may define a receiving lumen extending therethrough that is configured to receive the catheter sheath 12 and the dilation and capture assembly 14. Each of the catheter sheath 12 and the dilation and capture assembly 14 may be positioned within the receiving lumen of the introducer sheath 110 and including portions configured to extend distally from the introducer sheath 110 and be proximally retracted into the introducer sheath 110. Proximal portions of the catheter sheath 12 and the dilation and capture assembly 14 may extend proximally from the introducer sheath 110 to be positioned in an extracorporeal area 106 external to the body 104, and be physically manipulatable by a user. Distal portions of the catheter sheath 12 and the dilation and capture assembly 14 may extend distally from the introducer sheath 110 to be positioned within the body vessel 102 in an intracorporeal area 108 within the body 104 when a distal portion of the introducer sheath 110 is inserted into the body 104 of the patient. The introducer sheath 110 may be used to grip the catheter assembly 10 and perforate the body 104 of the patient so that the catheter sheath 12 and dilation and capture assembly 14 may be inserted into the body vessel 102. The introducer sheath 110 may first be inserted into the body 104 of the patient to create an access point for entry of the catheter sheath 12 and the dilation and capture assembly 14 into the body 104. While the depicted catheter assembly 10 includes the introducer sheath 110 for introducing the dilation and capture assembly 14 into the body 104 of the patient, it is contemplated and possible that the catheter assembly 10 does not include an introducer sheath, where the catheter sheath 12 and the dilation and capture assembly 14 may be inserted into the body 104 in combination or in another manner through other insertion devices.

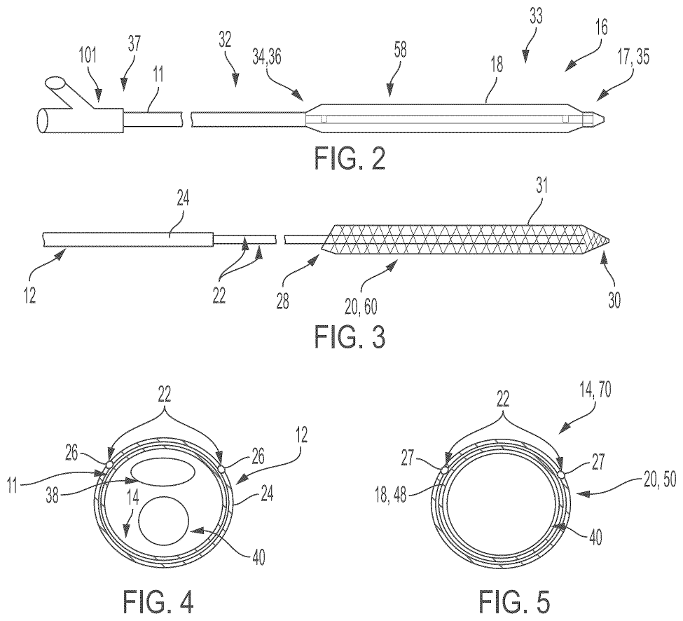

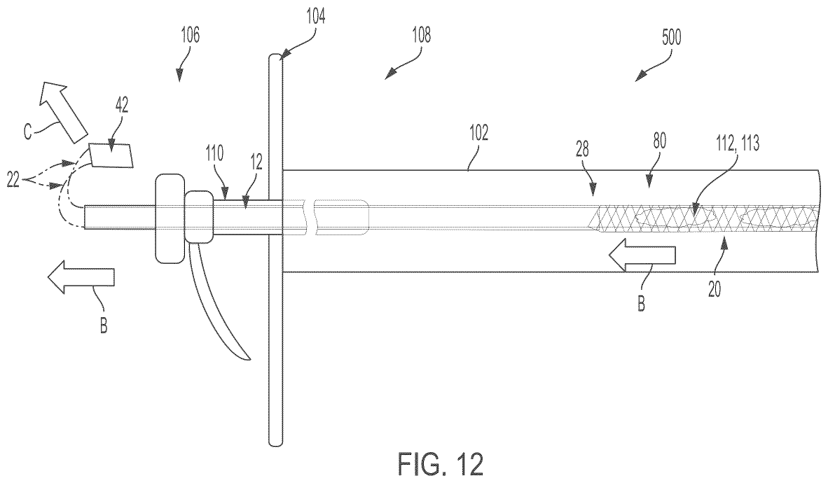

Referring to FIG. 1, the dilation and capture assembly 14 may include a catheter 16, a retrieval basket 20, and an actuator 42 (FIG. 12). As shown in FIG. 2, the catheter 16 may include a catheter wall 11, a hub 101, a proximal portion 32, a distal portion 33 positioned distal to the proximal portion 32, and a balloon portion 18 at the distal portion 33 and including a distal end 17 of the catheter 16. In FIG. 2, the balloon portion 18 is shown in an inflated position 58. The balloon portion 18 may be coupled to the catheter wall 11 at a distal end of and a position distal to the catheter wall 11. The catheter wall 11 may be an elongated, cylindrical body that extends between the hub 101 and the balloon portion 18 to connect the balloon portion 18 with the hub 101. The proximal portion 32 of the catheter 16 may include a proximal end 37 and an opposite distal end 36, and the balloon portion 18 of the distal portion 33 of the catheter 16 may include a proximal end 34 and a distal end 35 opposite the proximal end 34 of the balloon portion 18.

The balloon portion 18 may thus be positioned in the distal portion 33 distal to the proximal portion 32 with the distal end 36 of the proximal portion 32 of the catheter 16 being coupled to the proximal end 34 of the balloon portion 18 at the distal portion 33 of the catheter 16. The balloon portion 18 of the distal portion 33 may extend distally from the distal end 36 of the proximal portion 32. The hub 101 may be positioned proximal to the proximal portion 32 of the catheter 16 and coupled to the proximal end 37 of the proximal portion 32. When the dilation and capture assembly 14 is positioned within the body 104 of the patient, the hub 101 may extend proximally from the introducer sheath 110 so that the hub 101 may be physically manipulated by a user. The proximal portion 32 of the catheter 16 may be sized and shaped to be positioned and received within the catheter wall 11 of the catheter sheath 12.

The balloon portion 18 may be tapered at the distal end 35 and the proximal end 34 thereof. The balloon portion 18 may include an atraumatic tip at the distal end 35 to reduce damage and perforation to the body vessels 102 as the balloon portion 18 traverses the body vessels 102. In embodiments, the balloon portion 18 may be formed of a deformable material, such as polyurethane or silicone, to allow the balloon portion 18 to deform to maneuver through the body vessels 102.

Referring to FIG. 4, the catheter wall 11 of the catheter 16 may define an inflation lumen 38 and a guidewire lumen 40 extending therethrough. Particularly, the inflation lumen 38 and the guidewire lumen 40 may be defined within the catheter wall 11 at the proximal portion 32 of the catheter 16 and extend in parallel with one another. The inflation lumen 38 may be fluidly connected to the balloon portion 18 disposed at the distal end 17 of the catheter 16 and may be configured for inflating and deflating the balloon portion 18. The guidewire lumen 40 may extend from the proximal end 37 of the proximal portion 32 of the catheter 16 to the distal end 35 of the balloon portion 18 of the catheter 16 so that a guidewire (not shown) may be positioned within the catheter 16 and extend distally therefrom at the distal end 17. The guidewire lumen 40 may be configured to receive a guidewire for guiding the balloon portion 18, along with the rest of the dilation and capture assembly 14, through the body vessels 102. The balloon portion 18 may be a balloon on a balloon catheter, such as, for example, a Dorado™ PTA balloon catheter. In embodiments, the catheter 16 may include any other expansion device for performing a thrombectomy or an angioplasty instead of the balloon portion 18, such as, for example, an expandable cage, a stent, a stent graft, or other similar device configured for radial expansion.

Referring to FIG. 3, the retrieval basket 20 is shown in a deployed position 60. The retrieval basket 20 may include a retrieval side 28, a closed side 30 distally opposite the retrieval side 28, one or more attachment wire lumens 27 (FIG. 5), and one or more attachment wires 22 connecting the retrieval basket 20 to attachment wire lumens 26 (FIG. 4) of the catheter sheath 12. The one or more attachment wires 22 may be configured to position the retrieval basket 20 relative to the catheter sheath 12. The one or more attachment wires 22 may extend into the one or more attachment wire lumens 27 in the retrieval basket 20 to fixedly couple the one or more attachment wires 22 to the retrieval basket 20.

Referring to FIG. 4, the catheter sheath 12 may include a sheath wall 24 and one or more attachment wire lumens 26 defined by the sheath wall 24. The sheath wall 24 may be a hollow cylindrical wall configured to receive a portion of the dilation and capture assembly 14 within the sheath wall 24. However, the sheath wall 24 may be any operable shape capable of having the dilation and capture assembly 14 be positioned therein, such as, for example, a square or an oval. The one or more attachment wire lumens 26 may extend along a length of the sheath wall 24 and be configured to receive the attachment wires 22.

Referring to FIG. 5, the retrieval basket 20 may be formed of a mesh 31 configured to allow blood flow through the closed side 30 of the retrieval basket 20. The mesh 31 may be formed of any biocompatible material, such as, for example, nitinol. The mesh 31 may be sized so that particulate in the body vessel 102 from the thrombectomy may be collected by the retrieval basket 20. The retrieval side 28 of the retrieval basket 20 may be open so that particulate may enter and flow through the retrieval basket 20 to the closed side 30. The closed side 30 may be configured to retain particulate within the retrieval basket 20. The closed side 30 may be tapered or contoured to complement the tapering of the balloon portion 18. The closed side 30 may be deformable similar to the atraumatic tip of the balloon portion 18 to reduce damage to the body vessel 102 as the retrieval basket 20 is maneuvered through the body vessel 102. Referring to FIG. 5, in a delivery configuration 70, the retrieval basket 20 may be sized to be positioned around the balloon portion 18, with the balloon portion 18 at least partially positioned within the retrieval basket 20.

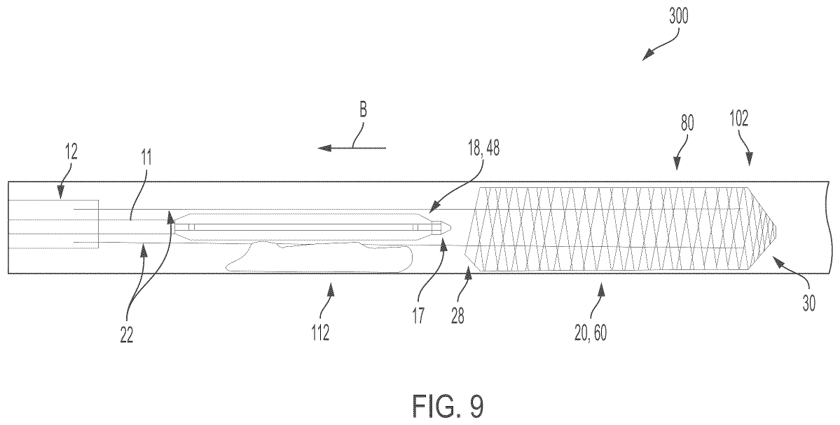

Referring to FIGS. 1-5, each of the attachment wires 22 may be slidably positioned within the one or more attachment wire lumens 26 to extend both proximally and distally from the catheter sheath 12. Referring to FIG. 3, each of the attachment wires 22 may extend distally from the attachment wire lumens 26 (FIG. 4) in the catheter sheath 12 to connect to attachment wire lumens 27 in the retrieval side 28 of the retrieval basket 20 and thus to connect the retrieval basket 20 to the catheter sheath 12. The retrieval basket 20 is configured to be moved between a delivery configuration 70 (FIGS. 1 and 5) to a retrieving configuration 80 (FIG. 9) as described in greater detail herein. The attachment wires 22 are fixedly coupled to the retrieval basket 20 and movably positioned within the attachment wire lumens 26 in the catheter sheath 12 so that the retrieval basket 20 is movable relative to the catheter sheath 12. The retrieval basket 20 is additionally movable relative to the balloon portion 18 so that the balloon portion 18 may move proximal to the retrieval basket 20. The retrieval basket 20 may be spaced apart from the catheter sheath 12 a distance that is greater than a length of the balloon portion 18 so that the balloon portion 18 may be moved to be positioned proximal to the retrieval basket 20 and distal to the catheter sheath 12.

The one or more attachment wires 22 may extend through the attachment wire lumens 27 to be connected to the closed side 30 of the retrieval basket 20. However, it is contemplated and possible that the attachment wires 22 are connected to the retrieval basket 20 at any position along the retrieval basket 20, such as a position at the retrieval side 28. The one or more attachment wires 22 may be formed of any biocompatible material capable of moving and retaining the retrieval basket 20 relative to the catheter sheath 12, such as, for example, nitinol.

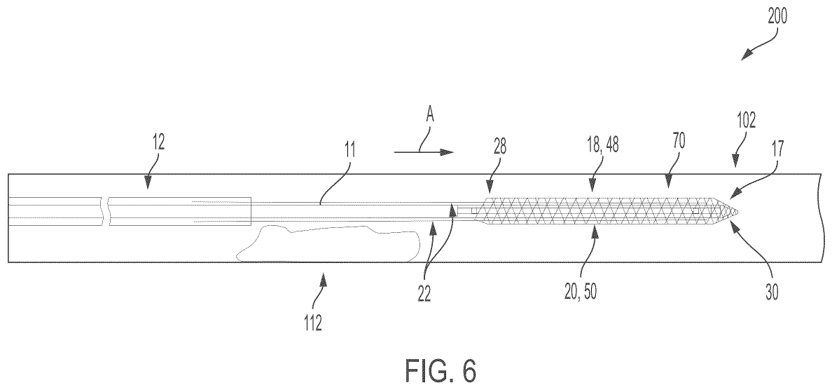

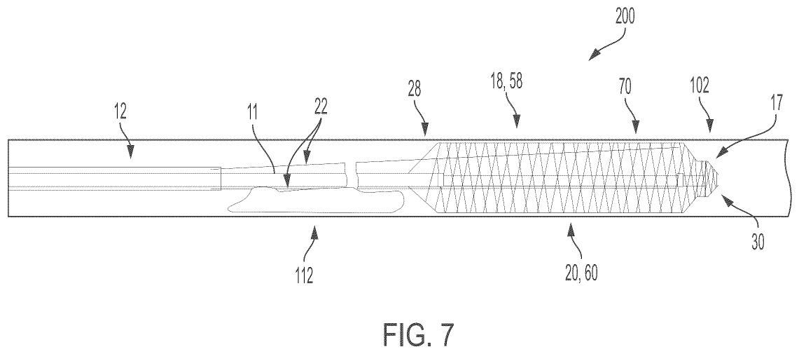

FIG. 6 depicts the catheter assembly 10 in the delivery configuration 70 past the thrombus site in a past thrombus position as a distally past thrombus site position 200 with the balloon portion 18 deflated and the retrieval basket 20 not deployed. FIG. 7 depicts the catheter assembly 10 in the distally past thrombus site position 200 with the balloon portion 18 inflated and the retrieval basket 20 deployed. In FIG. 7, the retrieval basket 20 of the catheter assembly 10 is in a delivery configuration 70. With reference to FIGS. 6 and 7, the balloon portion 18 may thus be selectively inflatable and deflatable between a deflated position 48 (FIG. 6) and an inflated position 58 (FIG. 7), respectively. The balloon portion 18 may be inflated and deflated via a fluid flowing through the inflation lumen 38. The fluid may be a saline and contrast mixture, such as used in an angiography. However, it is contemplated and possible that the fluid may be any fluid for inflating and deflating a balloon catheter, such as, for example, saline, water, air, a gas, or the like. The inflation lumen 38 may be in fluid connection with an fluid source (not shown) that controls fluid flow through the inflation lumen 38 and provides fluid to the balloon portion 18 through the inflation lumen 38 to inflate the balloon portion 18 to the inflated position 58, and draws fluid from the balloon portion 18 through the inflation lumen 38 to deflate the balloon portion 18 to the deflated position 48. The radius of the balloon portion 18 may be smaller in the deflated position 48 than the radius in the inflated position 58. The radius of the balloon portion 18 may be about the same as the radius of the body vessel 102 when in the inflated position 58. Referring to FIG. 6, in the deflated position 48, the balloon portion 18 may have a radius that is less than a radius of the catheter sheath 12 so that the balloon portion 18 may pass through the catheter sheath 12.

Further, the retrieval basket 20 may be movable between a collapsed position 50 (FIG. 6) and a deployed position 60 (FIG. 7). The retrieval basket 20 may be expanded from the collapsed position 50 to the deployed position 60 when the balloon portion 18 is inflated from the deflated position 48 to the inflated position 58 (FIG. 7). Particularly, the inflation of the balloon portion 18 from the deflated position 48 toward the inflated position 58 exerts pressure on the retrieval basket 20 to expand the retrieval basket 20 radially outward from the collapsed position 50 to the deployed position 60.

Referring to FIG. 10, the balloon portion 18 of the catheter 16 of the dilation and capture assembly 14 may be selectively inflatable and deflatable between a maceration orientation 59 and a delivery orientation 49. In the delivery orientation 49, the balloon portion 18 is deflated and the dilation and capture assembly 14 is configured to be inserted into a body vessel 102 of the body 104 of the patient. Particularly, in the delivery orientation 49, the balloon portion 18 is in the deflated position 48, and the retrieval basket 20 may be in the collapsed position 50. Referring again to FIG. 6, in the delivery configuration 70, the retrieval basket 20 may extend around the entirety of the balloon portion 18 that is in the deflated position 48 and be conformed to the shape of the balloon portion 18 so that the balloon portion 18 is in contact with the retrieval basket 20 along a length of the retrieval basket 20 between the closed side 30 and the retrieval side 28. With the retrieval basket 20 conforming to the shape of the balloon portion 18 in the deflated position 48, the dilation and capture assembly 14 is sized to be insertable within the body vessel 102 of the patient.

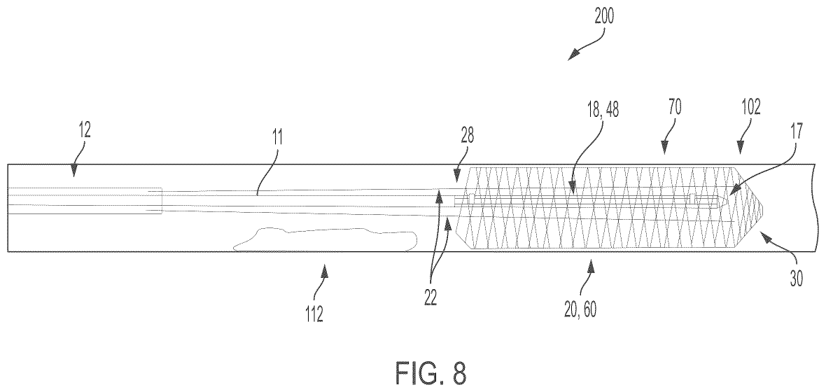

FIG. 8 depicts the catheter assembly 10 in a separated balloon and basket position 300 separating the balloon portion 18 and the retrieval basket 20 in the distally past thrombus site position 200 with the balloon portion 18 deflated in the deflated position 48 and the retrieval basket 20 deployed in the deployed position 60. FIG. 9 depicts the retrieval basket 20 in a retrieving configuration 80 in the distally past thrombus site position 200 with the balloon portion 18 proximally displaced to be at the thrombus 112. Referring again to FIGS. 6-9, the retrieval basket 20 may be deployable from the delivery configuration 70 (FIGS. 6-8) mounted to the balloon portion 18 to the retrieving configuration 80 (FIG. 9) axially displaced from the distal end 17 of the catheter 16.

Referring again to FIG. 6, in the delivery orientation 49 (FIG. 10), the balloon portion 18 is in the deflated position 48 to be sized so that the balloon portion 18 may be positioned within the retrieval basket 20 and move through the introducer sheath 110. With the retrieval basket 20 in the delivery configuration 70, the dilation and capture assembly 14 may be moved from the extracorporeal area 106 through the introducer sheath 110 and into the intracorporeal area 108 within the body vessel 102. Once the dilation and capture assembly 14 is positioned within the body vessel 102, the balloon portion 18 and retrieval basket 20 may be advanced in the direction of arrow A along the body vessel 102 to the distally past thrombus site position 200 positioned distal to the treatment site at the thrombus site position 100 where the thrombus 112 is located. The dilation and capture assembly 14 may be inserted so that the retrieval basket 20 is positioned distal to the thrombus 112 in the direction of blood flow so when the retrieval basket 20 is expanded to the deployed position 60, particulate removed from the thrombus 112 (such as via the balloon portion 18 as described herein to form the broken thrombus 113 as shown in FIG. 10) flows toward and into and/or may be captured by the retrieval side 28 of the retrieval basket 20 as described herein.

The balloon portion 18 may be expanded from the deflated position 48 (FIG. 6) to the inflated position 58 (FIG. 7) to have a radius that is approximately the size of the radius of the body vessel 102. As the balloon portion 18 expands from the deflated position 48 to the inflated position 58, the balloon portion 18 contacts the retrieval basket 20 to expand the retrieval basket 20 with the balloon portion 18 from the collapsed position 50 (FIG. 6) to the deployed position 60 (FIG. 7). When the retrieval basket 20 is in the deployed position 60, the retrieval basket 20 is expanded to the size of the body vessel 102 along with the balloon portion 18 so that any particulate later removed from the thrombus 112 as described herein by the balloon portion 18 flows into or is otherwise captured by the retrieval side 28 of the retrieval basket 20 when the balloon portion 18 is axially displaced from the retrieval basket 20. Once the retrieval basket 20 is dilated by the balloon portion 18, the mesh 31 is configured to maintain the size of the retrieval basket 20 so that the retrieval basket 20 does not reduce in size with the balloon portion 18 when the balloon portion 18 moves from the inflated position 58 to the deflated position 48 as shown in FIG. 8.

Referring to FIG. 8, once the retrieval basket 20 is in the deployed position 60 and dilated to the size of the body vessel 102, the dilation and capture assembly 14 may be moved to the separated balloon and basket position 300, where the balloon portion 18 is deflated from the inflated position 58 (FIG. 7) to the deflated position 48 (FIG. 8) with the retrieval basket 20 in the deployed position 60 so that the balloon portion 18 is separated, or spaced apart, from the retrieval basket 20.

Referring to FIG. 9, in the separated balloon and basket position 300 with the balloon portion 18 in the deflated position 48 and the retrieval basket 20 in the deployed position 60, the balloon portion 18 of the catheter 16 is further movable relative to the retrieval basket 20 such that the retrieval basket 20 changes to the retrieving configuration 80 axially displaced from the distal end 17 of the catheter 16. The hub 101 of the catheter 16 may be physically manipulated by a user to move the balloon portion 18 proximal to the retrieval basket 20 in the direction of arrow B so that the retrieval basket 20 is in the retrieving configuration 80. In the retrieving configuration 80, the retrieval basket 20 is positioned distal to the thrombus 112 with the balloon portion 18 positioned at the treatment site (e.g., the thrombus site position 100) between the catheter sheath 12 and the retrieval basket 20.

Referring to FIG. 10, the catheter 16 is shown with the balloon portion 18 in the deflated position 48 associated with the delivery orientation 49 after being in the inflated position 58 associated with the maceration orientation 59 to macerate and break the thrombus 112 to create the broken thrombus 113 (such as through multiple inflation/deflation of the balloon portion 18). Thus, when the balloon portion 18 is at the thrombus site position 100, the balloon portion 18 may be inflated to the inflated position 58 to contact the thrombus 112. Accordingly, when the retrieval basket 20 is in the retrieving configuration 80, the balloon portion 18 may be inflated from the delivery orientation 49 to a maceration orientation 59. When in the maceration orientation 59, the balloon portion 18 may interact and macerate the thrombus 112. In the maceration orientation 59, the balloon portion 18 may be repeatedly inflated and deflated between the inflated position 58 and the deflated position 48 to macerate the thrombus 112. The inflation of the balloon portion 18 may act to additionally perform an angioplasty by contacting and expanding the walls of the blood vessel 102, thereby dilating the blood vessel 102.

By moving between the deflated position 48 and the inflated position 58, the balloon portion 18 expands to contact and deform the thrombus 112, thereby breaking apart the thrombus 112 into broken thrombus 113 and dislodging the thrombus 112 from the body vessel 102. The balloon portion 18 may repeatedly move between the deflated position 48 and the inflated position 58 to reduce the size of or entirely remove the thrombus 112 from the body vessel 102. As the thrombus 112 breaks apart, particulate as broken thrombus 113 breaks apart from the thrombus 112 and flows through the body vessel 102 with the blood flow toward the retrieval basket 20. The broken thrombus 113 and the blood flowing through the body vessel 102 may enter the retrieval basket 20 at the retrieval side 28 and flow toward the closed side 30. As discussed above, the closed side 30 is sized to allow blood to flow through the closed side 30, while restricting movement of the broken thrombus 113. Accordingly, the closed side 30 of the retrieval basket 20 filters out particulate of the broken thrombus 113 from the blood, trapping the particulate of the broken thrombus 113 within the retrieval basket 20. The closed side 30 of the retrieval basket 20 may be shaped to prevent the collected particulate of the broken thrombus 113 from entirely restricting blood flow through the closed side 30. For example, the closed side 30 may be shaped as a conc.

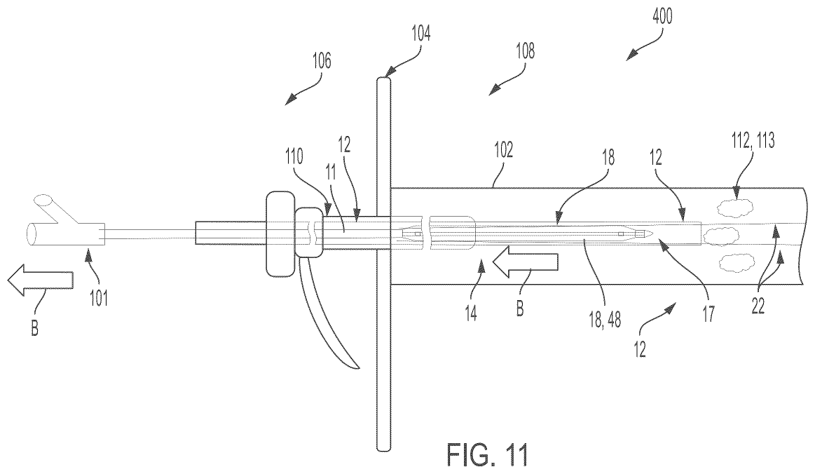

Referring to FIG. 11, the dilation and capture assembly 14 is depicted in a balloon removal position 400 in which the balloon portion 18 is proximally advanced from the thrombus site position 100 (previously including thrombus 112 at a treatment site). In the balloon removal position 400, the balloon portion 18 may be retracted in the direction of arrow B out of the body vessel 102. The balloon portion 18 is in the deflated position 48 so that the balloon portion 18 may be retracted through the catheter sheath 12 and the introducer sheath 110 from the intracorporeal area 108 to the extracorporeal area 106 to be removed from the body vessel 102. The hub 101 may be physically manipulated by a user to move the catheter 16 in the direction of arrow B. As the hub 101 is retracted in the direction of arrow B, the balloon portion 18 may pass through the introducer sheath 110 and the sheath wall 24 of the catheter sheath 12 without intervening with the attachment wires 22.

Referring to FIG. 12, the dilation and capture assembly 14 is depicted with the retrieval basket 20 in a retrieval basket removal position 500 and the retrieving configuration 80 to retrieve broken thrombus 113 when proximally being advanced back past the thrombus site including broken thrombus 113. The retrieval basket 20 is moved so that the retrieval side 28 of the retrieval basket 20 is proximally directed toward the catheter sheath 12. While moving toward the catheter sheath 12, the retrieval basket 20 may collect particulate of the broken thrombus 113 that is positioned between the retrieval basket 20 and the catheter sheath 12.

The retrieval basket 20 may be moved through the body vessel 102 toward the introducer sheath 110 in the direction of arrow B by operation of the actuator 42. Particularly, the actuator 42 may be operatively coupled to the attachment wires 22 where actuation of the actuator 42 pulls the attachment wires 22 through the attachment wire lumens 26 in the catheter sheath 12 in the direction of arrow B, thereby moving the retrieval basket 20 in the direction of arrow B. Actuation of the actuator 42 may include moving the actuator 42 in the direction of arrow C, where movement of the actuator 42 moves the attachment wires 22 along the attachment wire lumens 26, thereby pulling the retrieval basket 20 in the direction of arrow B toward the catheter sheath 12. The pulling of the retrieval basket 20 via the attachment wires 22 may cause the retrieval basket 20 to collapse from the deployed position 60 to the collapsed position 50.

As depicted, the actuator 42 may be a mechanical actuator, such as a tab, that the user may physically manipulate to move the retrieval basket 20. The actuator 42 may be positioned on the proximal portion 32 of the catheter 16 (FIG. 2) extending out of the introducer sheath 110 when the catheter 16 is positioned in the body vessel 102 so that the user can physically manipulate the actuator 42. However, it is contemplated and possible that the actuator 42 may be any actuator capable of moving the retrieval basket 20, such as, for example, a linear actuator, a rotary actuator, a pneumatic or hydraulic actuator, or the like. In such embodiments, the actuator 42 may include a motor to be automatically operated to move the attachment wires 22.

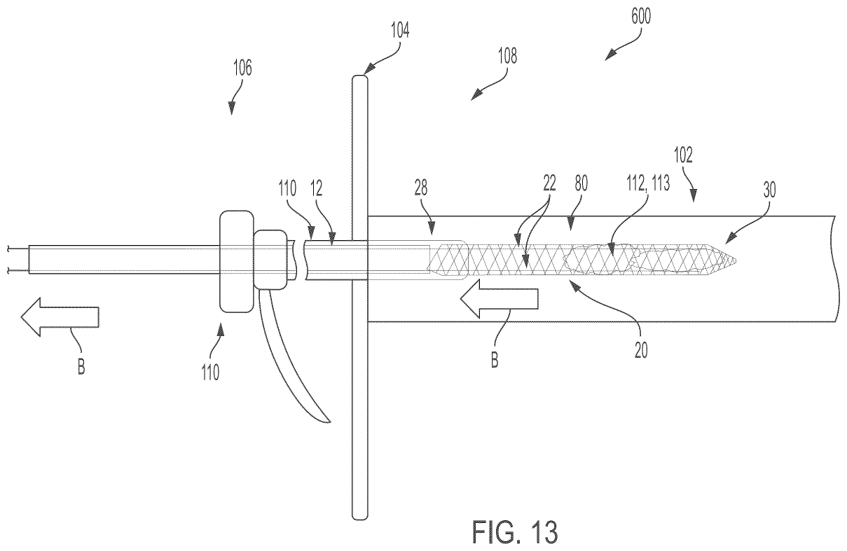

Referring to FIG. 13, the dilation and capture assembly 14 is depicted in an assembly extraction position 600. In the assembly extraction position 600, the retrieval basket 20 may be formed back into the collapsed position 50 so that the retrieval basket 20 is sized to be movable through the introducer sheath 110. Additionally, the contact between the catheter sheath 12 and the retrieval side 28 of the retrieval basket 20 while in the collapsed position 50 prevents particulate of the broken thrombus 113 trapped within the retrieval basket 20 from exiting the retrieval basket 20 through the retrieval side 28. In the assembly extraction position 600, the retrieval basket 20 may be moved, along with the particulate collected from maceration of the thrombus 112 and contained within the retrieval basket 20, in the direction of arrow B so that the catheter sheath 12 and the retrieval basket 20 may move from the intracorporeal area 108 through the introducer sheath 110 and out of the body vessel 102 to the extracorporeal area 106. The removal of the retrieval basket 20 from the body vessel 102 thereby removes the particulate of the broken thrombus 113 collected by the retrieval basket 20.

Thus a method of performing a thrombectomy as described herein may include inserting the catheter assembly 10 into the body vessel 102, as depicted in FIG. 1. As described above, the catheter assembly 10 may be inserted into the body vessel 102 through the introducer sheath 110 that extends from the extracorporeal area 106 into the intracorporeal area 108. The method further includes translating the balloon portion 18 and the retrieval basket 20 relative to the catheter sheath 12 in the direction of arrow A past the treatment site (e.g., the thrombus site position 100) so that the retrieval basket 20 is disposed distal to the thrombus 112, as depicted in FIG. 6.

As shown in FIG. 7, the method includes inflating the balloon portion 18 to the inflated position 58 as described herein, thereby dilating the retrieval basket 20 to the deployed position 60, as depicted in FIG. 7. As shown in FIG. 8, the balloon portion 18 may be deflated to the deflated position 48 so that the retrieval basket 20 is spaced apart from the balloon portion 18, and the balloon portion 18 is movable relative to the retrieval basket 20. As shown in FIG. 9, the balloon portion 18 may be translated in the direction of arrow B away from the retrieval basket 20 and toward the treatment site (e.g., the thrombus site position 100), so that the retrieval basket 20 is in the deployed position 60 and the balloon portion 18 is configured to contact the thrombus 112 when in the inflated position 58. As show in FIG. 10, once the balloon portion 18 is positioned at the treatment site (e.g., the thrombus site position 100), the balloon portion 18 is configured to be selectively and repeatedly inflatable and deflatable between respectively the maceration orientation 59 and the delivery orientation 49 to macerate the thrombus 112 and form the broken thrombus 113. The retrieval basket 20 in the retrieving configuration 80 is configured to collect the particulate of the broken thrombus 113 removed from the thrombus 112 during inflation and deflation of the balloon portion 18 at the treatment site (e.g., the thrombus site position 100).

As shown in FIG. 11, the balloon portion 18 may be deflated to the deflated position 48, so that the balloon portion 18 is sized to fit within the catheter sheath 12. As shown in FIG. 12, the balloon portion 18 may be retracted into the catheter sheath 12. The retrieval basket 20 may further be retracted proximally toward the treatment site (e.g., the thrombus site position 100) to collect the particulate of the broken thrombus 113 with the retrieval basket 20. The retrieval basket 20 may be retracted using the actuator 42, where actuation of the actuator 42 is configured to pull the attachment wires 22 and the retrieval basket 20 toward the treatment site. As shown in FIG. 13, the catheter assembly 10 may be retracted out of the body vessel 102 such as through the introducer sheath 110 thereby removing the particulate as the broken thrombus 113 from the body vessel 102.

Embodiments may be further described with reference to the following numbered clauses:

1. A catheter assembly for performing a thrombectomy, the catheter assembly including: a catheter sheath; and a dilation and capture assembly slidably positioned within the catheter sheath, the dilation and capture assembly including: a catheter including a balloon portion at a distal end of the catheter, the balloon portion being selectively inflatable and deflatable between respectively a maceration orientation and a delivery orientation; and a retrieval basket deployable from a delivery configuration mounted to the balloon portion and a retrieving configuration axially displaced from the distal end of the catheter.

2. The catheter assembly according to clause 1, wherein the retrieval basket comprises one or more attachment wires connecting the retrieval basket to the catheter sheath throughout movement of the retrieval basket from the delivery configuration to the retrieving configuration.

3. The catheter assembly according to clause 2, wherein: the catheter sheath comprises a sheath wall and one or more attachment wire lumens formed within the sheath wall; and the one or more attachment wires are slidably positioned within the one or more attachment wire lumens.

4. The catheter assembly according to any of clause 1 to clause 4, wherein: the retrieval basket comprises a retrieval side configured to receive particulate removed during the thrombectomy, and a closed side opposite the retrieval side configured to retain the particulate in the retrieval basket, and the retrieval basket is at least partially formed of a mesh configured to allow blood flow through the closed side of the retrieval basket.

5. The catheter assembly according to clause 4, wherein: the retrieval basket comprises one or more attachment wires connecting the retrieval side of the retrieval basket to the catheter sheath throughout movement of the retrieval basket from the delivery configuration to the retrieving configuration.

6. The catheter assembly according to clause 5, wherein: the catheter sheath comprises a sheath wall and one or more attachment wire lumens formed within the sheath wall; and the one or more attachment wires are slidably positioned within the one or more attachment wire lumens.

7. The catheter assembly according to any of clause 5 or clause 6, wherein the one or more attachment wires are formed of nitinol.

8. The catheter assembly according to any of clause 4 to clause 7, wherein: the catheter comprises a proximal portion coupled to a proximal end of the balloon portion, the catheter defining an inflation lumen for inflating and deflating the balloon portion and a guidewire lumen.

9. The catheter assembly according to any of clause 1 to clause 8, further including: an actuator operatively coupled to the retrieval basket, the actuator being configured to axially translate the retrieval basket.

10. A catheter assembly for performing an thrombectomy, the catheter assembly including: a catheter sheath; a dilation and capture assembly slidably positioned within the catheter sheath, the dilation and capture assembly including: a catheter including a proximal portion and a balloon portion at a distal end of the proximal portion, the balloon portion being selectively inflatable and deflatable between respectively a maceration orientation and a delivery orientation; a retrieval basket deployable from a delivery configuration mounted to the balloon portion and a retrieving configuration axially displaced from the balloon portion; and one or more attachment wires configured to position the retrieval basket relative to the catheter sheath.

11. The catheter assembly according to clause 10, wherein: the catheter sheath comprises a sheath wall and one or more attachment wire lumens formed within the sheath wall; and the one or more attachment wires are slidably positioned within the one or more attachment wire lumens.

12. The catheter assembly according to any of clause 10 or clause 11, wherein: the retrieval basket comprises a retrieval side configured to receive particulate removed during the thrombectomy, and a closed side opposite the retrieval side configured to retain the particulate in the retrieval basket, and the retrieval basket is at least partially formed of a mesh configured to allow blood flow through the closed side of the retrieval basket.

13. The catheter assembly according to clause 12, wherein: the one or more attachment wires connect the retrieval side of the retrieval basket to the catheter sheath throughout movement of the retrieval basket from the delivery configuration to the retrieving configuration.

14. The catheter assembly according to any of clause 12 or clause 13, wherein: the catheter defines an inflation lumen for inflating and deflating the balloon portion and a guidewire lumen.

15. The catheter assembly according to any of clause 10 to clause 14, further including an actuator operatively coupled to the retrieval basket, the actuator being configured to axially translate the retrieval basket.

16. A method of performing a thrombectomy, the method including: inserting a catheter assembly into a body vessel, the catheter assembly including: a catheter sheath; and a dilation and capture assembly slidably positioned within the catheter sheath, the dilation and capture assembly including: a catheter including a balloon portion at a distal end of the catheter, the balloon portion being selectively inflatable and deflatable between respectively a maceration orientation and a delivery orientation; and a retrieval basket deployable from a delivery configuration mounted to the balloon portion and a retrieving configuration axially displaced from the distal end of the catheter; translating the balloon portion and the retrieval basket relative to the catheter sheath past a treatment site; and inflating the balloon portion, thereby dilating the retrieval basket.

17. The method according to clause 16, further including: deflating the balloon portion; translating the balloon portion toward the treatment site; repeatedly inflating and deflating the balloon portion at the treatment site.

18. The method according to clause 17, wherein the retrieval basket collects particulate removed during inflation and deflation of the balloon portion at the treatment site.

19. The method according to clause 18, further including: deflating the balloon portion; and retracting the balloon portion into the catheter sheath.

20. The method according to clause 19, further including: retracting the retrieval basket toward the treatment site to collect the particulate with the retrieval basket; and retracting the catheter assembly out of the body vessel.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter assembly for performing a thrombectomy, the catheter assembly comprising:
a catheter sheath; and
a dilation and capture assembly slidably positioned within the catheter sheath, the dilation and capture assembly comprising:
a catheter comprising a balloon portion at a distal end of the catheter, the balloon portion being selectively inflatable and deflatable between respectively a maceration orientation and a delivery orientation; and
a retrieval basket deployable from a delivery configuration mounted to the balloon portion and a retrieving configuration axially displaced from the distal end of the catheter.

2. The catheter assembly of claim 1, wherein the retrieval basket comprises one or more attachment wires connecting the retrieval basket to the catheter sheath throughout movement of the retrieval basket from the delivery configuration to the retrieving configuration.

3. The catheter assembly of claim 2, wherein:
the catheter sheath comprises a sheath wall and one or more attachment wire lumens formed within the sheath wall; and
the one or more attachment wires are slidably positioned within the one or more attachment wire lumens.

4. The catheter assembly of claim 1, wherein:
the retrieval basket comprises a retrieval side configured to receive particulate removed during the thrombectomy, and a closed side opposite the retrieval side configured to retain the particulate in the retrieval basket, and
the retrieval basket is at least partially formed of a mesh configured to allow blood flow through the closed side of the retrieval basket.

5. The catheter assembly of claim 4, wherein:
the retrieval basket comprises one or more attachment wires connecting the retrieval side of the retrieval basket to the catheter sheath throughout movement of the retrieval basket from the delivery configuration to the retrieving configuration.

6. The catheter assembly of claim 5, wherein:
the catheter sheath comprises a sheath wall and one or more attachment wire lumens formed within the sheath wall; and
the one or more attachment wires are slidably positioned within the one or more attachment wire lumens.

7. The catheter assembly of claim 5, wherein the one or more attachment wires are formed of nitinol.

8. The catheter assembly of claim 4, wherein:
the catheter comprises a proximal portion coupled to a proximal end of the balloon portion, the catheter defining an inflation lumen for inflating and deflating the balloon portion and a guidewire lumen.

9. The catheter assembly of claim 1, further comprising:
an actuator operatively coupled to the retrieval basket, the actuator being configured to axially translate the retrieval basket.

10. A catheter assembly for performing a thrombectomy, the catheter assembly comprising:
a catheter sheath; and
a dilation and capture assembly slidably positioned within the catheter sheath, the dilation and capture assembly comprising:
a catheter comprising a proximal portion and a balloon portion at a distal end of the proximal portion, the balloon portion being selectively inflatable and deflatable between respectively a maceration orientation and a delivery orientation;
a retrieval basket deployable from a delivery configuration mounted to the balloon portion and a retrieving configuration axially displaced from the balloon portion; and
one or more attachment wires configured to position the retrieval basket relative to the catheter sheath.

11. The catheter assembly of claim 10, wherein:
the catheter sheath comprises a sheath wall and one or more attachment wire lumens formed within the sheath wall; and
the one or more attachment wires are slidably positioned within the one or more attachment wire lumens.

12. The catheter assembly of claim 10, wherein:
the retrieval basket comprises a retrieval side configured to receive particulate removed during the thrombectomy, and a closed side opposite the retrieval side configured to retain the particulate in the retrieval basket, and
the retrieval basket is at least partially formed of a mesh configured to allow blood flow through the closed side of the retrieval basket.

13. The catheter assembly of claim 12, wherein:
the one or more attachment wires connect the retrieval side of the retrieval basket to the catheter sheath throughout movement of the retrieval basket from the delivery configuration to the retrieving configuration.

14. The catheter assembly of claim 12, wherein:
the catheter defines an inflation lumen for inflating and deflating the balloon portion and a guidewire lumen.

15. The catheter assembly of claim 10, further comprising an actuator operatively coupled to the retrieval basket, the actuator being configured to axially translate the retrieval basket.

16. A method of performing a thrombectomy, the method comprising:
inserting a catheter assembly into a body vessel, the catheter assembly comprising:
a catheter sheath; and
a dilation and capture assembly slidably positioned within the catheter sheath, the dilation and capture assembly comprising:
a catheter comprising a balloon portion at a distal end of the catheter, the balloon portion being selectively inflatable and deflatable between respectively a maceration orientation and a delivery orientation; and
a retrieval basket deployable from a delivery configuration mounted to the balloon portion and a retrieving configuration axially displaced from the distal end of the catheter;
translating the balloon portion and the retrieval basket relative to the catheter sheath past a treatment site; and
inflating the balloon portion, thereby dilating the retrieval basket.

17. The method of claim 16, further comprising:
deflating the balloon portion;
translating the balloon portion toward the treatment site; and
repeatedly inflating and deflating the balloon portion at the treatment site.

18. The method of claim 17, wherein the retrieval basket collects particulate removed during inflation and deflation of the balloon portion at the treatment site.

19. The method of claim 18, further comprising:
deflating the balloon portion; and
retracting the balloon portion into the catheter sheath.

20. The method of claim 19, further comprising:
retracting the retrieval basket toward the treatment site to collect the particulate with the retrieval basket; and
retracting the catheter assembly out of the body vessel.
